(19) United States
(12) Patent Application Publication
(10) Pub. No.: US 2013/0150918 A1
(43) Pub. Date: Jun. 13, 2013

(54) SYSTEM AND METHOD FOR AUTOMATICALLY TRAINING A NEUROSTIMULATION SYSTEM

(71) Applicant: Boston Scientific Neuromodulation Corporation, Valencia, CA (US)

(72) Inventors: David K.L. Peterson, Valencia, CA (US); Jordi Parramon, Valencia, CA (US); Kerry Bradley, Glendale, CA (US); Michael A. Moffitt, Valencia, CA (US)

(73) Assignee: BOSTON SCIENTIFIC NEUROMODULATION CORPORATION, Valencia, CA (US)

(21) Appl. No.: 13/707,376

(22) Filed: Dec. 6, 2012

Related U.S. Application Data
(60) Provisional application No. 61/568,600, filed on Dec. 8, 2011.

(51) Int. Cl. A61N 1/08; A61N 1/36

(52) U.S. Cl. CPC A61N 1/08; A61N 1/36071
USPC 607/46; 607/59

(57) ABSTRACT

Neurostimulators, neurostimulation systems, and methods for providing therapy to a patient. A neurostimulation system stores reference measurements and reference stimulation parameter sets respectively associated with the reference measurements. A new measurement of least one environmental parameter indicative of a change in a therapeutic environment is taken. Whether the new measurement matches one of the stored reference measurements is determined. If a match is determined, stimulation energy is conveyed from the neurostimulation system to the patient in accordance with the stimulation parameter set corresponding to the matching reference measurement. If a match is not determined, stimulation energy is conveyed from the neurostimulation system to the patient in accordance with a user-defined stimulation parameter set, another reference stimulation parameter set is defined based on the user-defined stimulation parameter set, and the new measurement is stored as an additional reference measurement in association with the additional reference stimulation parameter set.

### RELATED APPLICATION DATA

The present application claims the benefit under 35 U.S.C. §119 to U.S. provisional patent application Ser. No. 61/568,600, filed Dec. 8, 2011. The foregoing application is hereby incorporated by reference into the present application in its entirety.

### FIELD OF THE INVENTION

The present invention relates to tissue stimulation systems, and more particularly, to apparatus and methods for programming neurostimulation systems.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, Functional Electrical Stimulation (FES) systems such as the Freehand system by NeuroControl (Cleveland, Ohio) have been applied to restore some functionality to paralyzed extremities in spinal cord injury patients. Furthermore, in recent investigations Peripheral Nerve Stimulation (PNS) systems have demonstrated efficacy in the treatment of chronic pain syndromes and incontinence, and a number of additional applications are currently under investigation. Occipital Nerve Stimulation (ONS), in which leads are implanted in the tissue over the occipital nerves, has shown promise as a treatment for various headaches, including migraine headaches, cluster headaches, and cervicogenic headaches.

These implantable neurostimulation systems typically include one or more electrode carrying neurostimulation leads, which are implanted at the desired stimulation site, and a neurostimulator (e.g., an implantable pulse generator (IPG)) implanted remotely from the stimulation site, but coupled either directly to the neurostimulation lead(s) or indirectly to the neurostimulation lead(s) via a lead extension. Thus, electrical pulses can be delivered from the neurostimulator to the neurostimulation leads to stimulate the tissue and provide the desired efficacious therapy to the patient.

The neurostimulation system may further comprise an external control device to remotely instruct the neurostimulator to generate electrical stimulation pulses in accordance with selected stimulation parameters. For example, the neurostimulation system may further comprise a handheld patient programmer in the form of a remote control (RC) to remotely instruct the neurostimulator to generate electrical stimulation pulses in accordance with the selected stimulation parameters. The RC may, itself, be programmed by a clinician, for example, by using a computerized programming system in the form of a clinician's programmer (CP), which typically includes a general purpose computer, such as a laptop, with a programming software package installed thereon.

Electrical stimulation energy may be delivered from the neurostimulator to the electrodes in the form of an electrical pulsed waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, width (or duration), and frequency (or rate) of the electrical pulses provided through the electrode array. Each electrode combination, along with the electrical pulse parameters, can be referred to as a “stimulation parameter set.”

With some neurostimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode combinations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit, while minimizing the volume of non-target tissue that is stimulated.

However, the number of electrodes available, combined with the ability to generate a variety of complex stimulation pulses, presents a huge selection of stimulation parameter sets to the clinician or patient. For example, if the neurostimulation system to be programmed has an array of sixteen electrodes, millions of stimulation parameter sets may be available for programming into the neurostimulation system. Today, neurostimulation system may have up to thirty-two electrodes, thereby exponentially increasing the number of stimulation parameters sets available for programming.

To facilitate such selection, the clinician generally programs the neurostimulator through a computerized programming system, such as the afore-described CP. This programming system can be a self-contained hardware/software system, or can be defined predominantly by software running on a standard personal computer (PC). The PC or custom hardware may actively control the characteristics of the electrical stimulation generated by the neurostimulator to allow the optimum stimulation parameters to be determined based on patient feedback or other means and to subsequently program the neurostimulator with the optimum stimulation parameter set or sets. The computerized programming system may be operated by a clinician attending the patient in several scenarios.

In order to achieve an effective result, the lead or leads must be placed in a location, such that the electrical stimulation will effectively treat the indentified disease or condition. If a lead is not correctly positioned, it is possible that the patient will receive little or no benefit from the implanted neurostimulator. Thus, correct lead placement can mean the difference between effective and ineffective pain therapy. When electrical leads are implanted within the patient, the computerized programming system, in the context of an operating room (OR) mapping procedure, may be used to instruct the neurostimulator to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the neurostimulator, with a set of stimulation parameters that best addresses the disease or condition. Thus, the navigation session may be used to pinpoint the stimulation region or areas correlating to the disease or condition. Such programming ability is particularly advantageous for targeting the tissue during implantation, or after implantation should the leads gradually or unexpectedly move that would otherwise relocate the stimulation energy away from the target site. Such migration of leads relative to each other or relative to tissue may be caused by postural changes made by the patient (e.g., standing up, lying down, trunk twisting, bending, etc.). By reprogramming the neurostimulator (typically by independently varying the stimulation energy on the electrodes), the stimulation region can often be moved back to the effective pain site without having to re-operate on the patient in order to reposition the lead and its electrode array.

Some neurostimulation systems are capable of automatically adjusting the programming of the neurostimulator based on input measurements (e.g., impedance measurements, electrical field measurements, accelerometers, etc.) indicative of movement of the lead(s) relative to each other or tissue. These neurostimulation systems must be calibrated by establishing a fit between the input measurements and the associated efficacious and comfortable stimulation parameter sets. This might be accomplished by having the patient assume different postures (e.g., sitting, standing, laying down, etc.) to effect different input measurements and then adjusting the stimulation until an efficacious and comfortable stimulation parameter set is achieved for each posture. After calibration, the neurostimulation system may adjust the stimulation in response to the input measurements in accordance with the fitting process. Subsequent changes in the relationships between the input measurements and the stimulation parameter sets require a new calibration process.

There, thus, remains a need for a more robust technique for calibrating a neurostimulation system that automatically adjusts stimulation to maintain efficacious and comfortable therapy of the patient.

### SUMMARY OF THE INVENTION

In accordance with a first aspect of the present inventions, a neurostimulator comprises input/output circuitry configured for receiving a user-defined stimulation parameter set from an external control device, stimulation output circuitry configured for conveying electrical stimulation energy, and monitoring circuitry configured for acquiring a new measurement of least one environmental parameter indicative of a change in a therapeutic environment. The environmental parameter(s) can comprise, e.g., an impedance, field potential, evoked potential, pressure, translucence, reflectance, pH, acceleration, chemical, neural recordings, and time of day. The neurostimulator further comprises memory configured for storing a plurality of reference measurements and a plurality of reference stimulation parameter sets respectively associated with the reference measurements. Each of the reference stimulation parameter sets may comprise, e.g., a stimulation energy intensity and/or an electrode combination.

The neurostimulator further comprises a controller configured for determining whether the new measurement matches one of the stored reference measurements. If a match is determined, the controller is further configured for instructing the stimulation output circuitry to convey electrical stimulation energy in accordance with the stimulation parameter set corresponding to the matching reference measurement. If a match is not determined, the controller is configured for instructing the stimulation output circuitry to convey electrical stimulation energy in accordance with the user-defined stimulation parameter set, defining another reference stimulation parameter set based on the user-defined stimulation parameter set, and storing the new measurement as an additional reference measurement in the memory in association with the additional reference stimulation parameter set.

In one embodiment, the user-defined stimulation parameter set is defined as the additional reference stimulation parameter set. In this case, the user-defined stimulation parameter set can be compared to a threshold, and the user-defined stimulation parameter defined as the additional reference stimulation parameter set only if the user-defined stimulation parameter set does not exceed the threshold.

In another embodiment, the monitoring circuitry is further configured for acquiring a plurality of new measurements, each of least one environmental parameter indicative of a change in a therapeutic environment. In this case, the controller may be configured for storing the additional reference stimulation parameter set only if the new measurements are substantially the same during the conveyance of the electrical stimulation in accordance with the user-defined stimulation parameter set. The plurality of new measurements may be a predetermined number. An optional embodiment of the neurostimulator may comprise a housing containing the input/output circuitry, stimulation output circuitry, memory, and controller.

In accordance with a second aspect of the present inventions, a neurostimulation system is provided. The neurostimulation system comprises an external control device configured for allowing a user to define a stimulation parameter set and a neurostimulator configured for acquiring a new measurement of least one environmental parameter indicative of a change in a therapeutic environment, storing a plurality of reference measurements and a plurality of reference stimulation parameter sets respectively associated with the reference measurements, and determining whether the new measurement matches one of the stored reference measurements. If a match is determined, the neurostimulator is configured for conveying electrical stimulation energy in accordance with the stimulation parameter set corresponding to the matching reference measurement. If a match is not determined, the neurostimulator is configured for conveying electrical stimulation energy in accordance with the user-defined stimulation parameter set, defining another reference stimulation parameter set based on the user-defined stimulation parameter set, and storing the new measurement as an additional reference measurement in the memory in association with the additional reference stimulation parameter set.

The environmental parameter(s) can comprise, e.g., an impedance, field potential, evoked potential, pressure, translucence, reflectance, pH, acceleration, chemical, neural recordings, and time of day. Each of the reference stimulation parameter sets may comprise, e.g., a stimulation energy intensity and/or an electrode combination. In one embodiment, the external control device is configured for defining the user-defined stimulation parameter set as the additional reference stimulation parameter set. In this case, the neurostimulator may be further configured for comparing the user-defined stimulation parameter set to a threshold, and defining the user-defined stimulation parameter as the additional reference stimulation parameter set only if the user-defined stimulation parameter set does not exceed the threshold. In another embodiment, the neurostimulator is further configured for acquiring a plurality of new measurements, each of least one environmental parameter indicative of a change in a therapeutic environment, and storing the additional reference stimulation parameter set only if the plurality of new measurements are substantially the same during the conveyance of the electrical stimulation in accordance with the user-defined stimulation parameter set. The plurality of new measurements may be a predetermined number.

In accordance with a third aspect of the present inventions, a method of operating a neurostimulation system to provide therapy (e.g., treatment of chronic pain) to a patient via electrical stimulation energy is provided. The neurostimulation system is configured for storing a plurality of reference measurements and a plurality of reference stimulation parameter sets respectively associated with the reference measurements. Each of the reference stimulation parameter sets may comprise, e.g., at least one of a stimulation energy intensity and an electrode combination. The method comprises taking a new measurement of least one environmental parameter indicative of a change in a therapeutic environment. The environmental parameter(s) can comprise, e.g., an impedance, field potential, evoked potential, pressure, translucence, reflectance, pH, acceleration, chemical, neural recordings, and time of day.

The method further comprises determining whether the new measurement matches one of the stored reference measurements. If a match is determined, electrical stimulation energy is conveyed from the neurostimulation system to the patient in accordance with the stimulation parameter set corresponding to the matching reference measurement. If a match is not determined, electrical stimulation energy is conveyed from the neurostimulation system to the patient in accordance with a user-defined stimulation parameter set, another reference stimulation parameter set is defined based on the user-defined stimulation parameter set, and the new measurement is stored as an additional reference measurement in association with the additional reference stimulation parameter set.

One method comprises defining the user-defined stimulation parameter set as the additional reference stimulation parameter set. In this case, the method may further comprise comparing the user-defined stimulation parameter set to a threshold, and the user-defined stimulation parameter is only defined as the additional reference stimulation parameter set if the user-defined stimulation parameter set does not exceed the threshold. Another method further comprising taking a plurality of new measurements, each of least one environmental parameter indicative of a change in a therapeutic environment. In this case, the additional reference stimulation parameter set is only stored if the new measurements are substantially the same during the conveyance of the electrical stimulation in accordance with the user-defined stimulation parameter set. The plurality of new measurements may be a predetermined number.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of one embodiment of a spinal cord stimulation (SCS) system arranged in accordance with the present inventions;

FIG. 2 is a plan view of the SCS system of FIG. 1 in use with a patient;

FIG. 3 is a plan view of an implantable pulse generator (IPG) and an embodiment of a percutaneous stimulation lead used in the SCS system of FIG. 1;

FIG. 4 is a block diagram of the internal componentry of the implantable pulse generator of FIG. 1;

FIG. 5 is a plan view of a remote control that can be used in the SCS system of FIG. 1;

FIG. 6 is a block diagram of the internal componentry of the remote control of FIG. 5;

FIG. 7 is a timing diagram illustrating an exemplary variance in the distance between stimulating electrodes and a nerve over time;

FIG. 8 is a timing diagram illustrating an exemplary manual adjustment in stimulation energy in response to the variance in the distance between stimulating electrodes and a nerve over time;

FIG. 9 is one method of training an automated stimulation adjustment algorithm used in the RC of the SCS system of FIG. 1; and

FIG. 10 is a timing diagram illustrating exemplary training of the automated stimulation adjustment algorithm in response to a manual adjustment in stimulation energy.

### DETAILED DESCRIPTION OF THE EMBODIMENTS

The description that follows relates to a spinal cord stimulation (SCS) system. However, it is to be understood that the while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

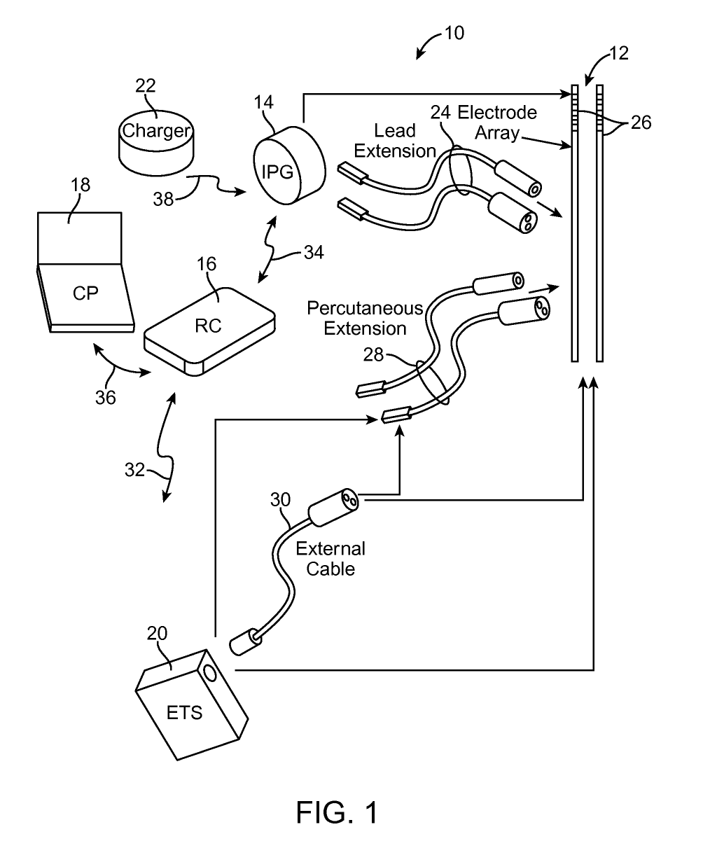

Turning first to FIG. 1, an exemplary SCS system 10 generally includes a plurality (in this case, two) of implantable neurostimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. The number of neurostimulation leads 12 illustrated is two, although any suitable number of neurostimulation leads 12 can be provided, including only one. Alternatively, a surgical paddle lead in can be used in place of one or more of the percutaneous leads. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Thus, any functions described herein with respect to the IPG 14 can likewise be performed with respect to the ETS 20. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

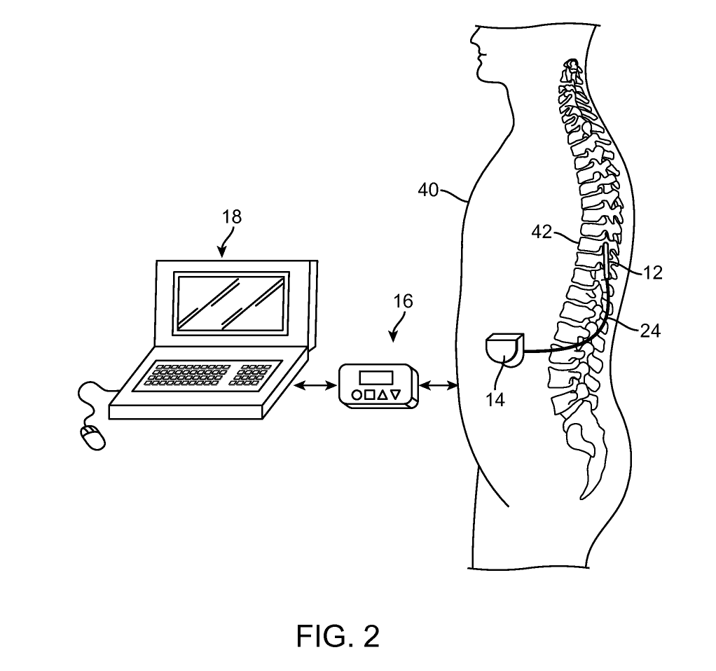

As shown in FIG. 2, the neurostimulation leads 12 are implanted within the spinal column 42 of a patient 40. The preferred placement of the neurostimulation leads 12 is adjacent, i.e., resting upon, the spinal cord area to be stimulated. Due to the lack of space near the location where the neurostimulation leads 12 exit the spinal column 42, the IPG 14 is generally implanted in a surgically-made pocket either in the abdomen or above the buttocks. The IPG 14 may, of course, also be implanted in other locations of the patient's body. The lead extension 24 facilitates locating the IPG 14 away from the exit point of the neurostimulation leads 12. As there shown, the CP 18 communicates with the IPG 14 via the RC 16.

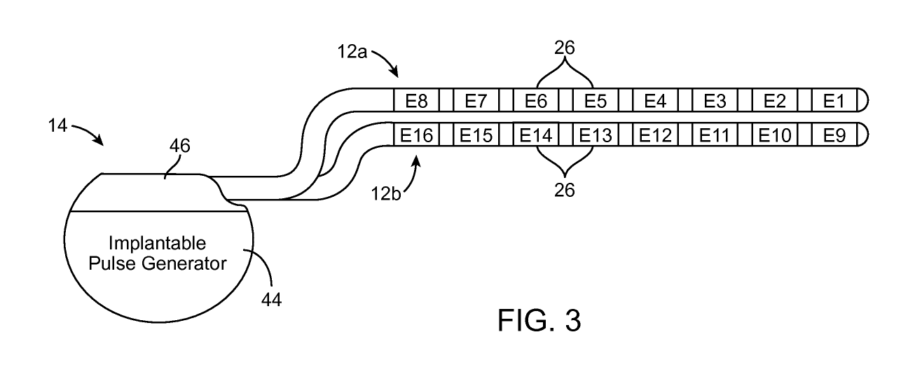

Referring now to FIG. 3, the external features of the neurostimulation leads 12 and the IPG 14 will be briefly described. One of the neurostimulation leads 12a has eight electrodes 26 (labeled E1-E8), and the other stimulation lead 12b has eight electrodes 26 (labeled E9-E16). The actual number and shape of leads and electrodes will, of course, vary according to the intended application. The IPG 14 comprises an outer case 44 for housing the electronic and other components (described in further detail below), and a connector 46 to which the proximal ends of the neurostimulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode configurations, which define the electrodes that are activated as anodes (positive), cathodes (negative), and turned off (zero), percentage of stimulation energy assigned to each electrode (fractionalized electrode configurations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the case of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

The IPG 14 is capable of taking measurements that are indicative of the coupling efficiencies between the electrode array 26 and the surrounding tissue. Notably, in the case of SCS, the electrode array 26 fits snugly within the epidural space of the spinal column 42, and because the tissue is conductive, there is an impedance associated therewith that indicates how easily current flows therethrough. Thus, the electrode impedance can be measured in order to determine the coupling efficiency between the respective electrode array 26 and the tissue. Other electrical parameter data, such as field potential and evoked action potential, may also be measured to ultimately determine the coupling efficiency between the electrodes 26 and the tissue.

Electrical data can be measured using any one of a variety means. For example, the electrical data measurements can be made on a sampled basis during a portion of the time while the electrical stimulus pulse is being applied to the tissue, or immediately subsequent to stimulation, as described in U.S. patent application Ser. No. 10/364,436, which has previously been incorporated herein by reference. Alternatively, the electrical data measurements can be made independently of the electrical stimulation pulses, such as described in U.S. Pat. Nos. 6,516,227 and 6,993,384, which are expressly incorporated herein by reference. For example, electrical data measurements can be made in response to alternating current (AC) or pulsatile electrical signals, which preferably use amplitudes and pulsewidths (e.g., 1 mA for 20 μs) that generate no physiological response for the patient (i.e., subthreshold), but can alternatively be performed in response to stimulation pulses.

The impedance measurement technique may be performed by measuring impedance vectors, which can be defined as impedance values measured between selected pairs of electrodes 26. The interelectrode impedance may be determined in various ways. For example, a known current (in the case where the IPG 14 is sourcing current) can be applied between a pair of electrodes 26, a voltage between the electrodes 26 can be measured, and an impedance between the electrodes 26 can be calculated as a ratio of the measured voltage to known current. Or a known voltage (in the case where the IPG is sourcing voltage) can be applied between a pair of electrodes 26, a current between the electrodes 26 can be measured, and an impedance between the electrodes 26 can be calculated as a ratio of the known voltage to measured current.

The field potential measurement technique may be performed by generating an electrical field at selected ones of the electrodes 26 and recording the electrical field at other selected ones of the lead electrodes 26. This may be accomplished in one of a variety of manners. For example, an electrical field may be generated conveying electrical energy to a selected one of the electrodes 26 and returning the electrical energy at the IPG case. Alternatively, multipolar configurations (e.g., bipolar or tripolar) may be created between the lead electrodes 26. Or, an electrode that is sutured (or otherwise permanently or temporarily attached (e.g., an adhesive or gel-based electrode) anywhere on the patient's body may be used in place of the case IPG outer case or lead electrodes 26. In either case, while a selected one of the electrodes 26 is activated to generate the electrical field, a selected one of the electrodes 26 (different from the activated electrode) is operated to record the voltage potential of the electrical field.

The evoked potential measurement technique may be performed by generating an electrical field at one of the electrodes 26, which is strong enough to depolarize the neurons adjacent the stimulating electrode beyond a threshold level, thereby inducing the firing of action potentials (APs) that propagate along the neural fibers. Such stimulation is preferably supra-threshold, but not uncomfortable. A suitable stimulation pulse for this purpose is, for example, 4 mA for 200 μs. While a selected one of the electrodes 26 is activated to generate the electrical field, a selected one or ones of the electrodes 26 (different from the activated electrode) is operated to record a measurable deviation in the voltage caused by the evoked potential due to the stimulation pulse at the stimulating electrode.

Further details discussing the measurement of electrical parameter data, such as electrode impedance, field potential, and evoked action potentials, as well as other parameter data, such as pressure, translucence, reflectance and pH (which can alternatively be used), to determine the coupling efficiency between an electrode and tissue are set forth in U.S. patent application Ser. No. 10/364,436, entitled “Neural Stimulation System Providing Auto Adjustment of Stimulus Output as a Function of Sensed Impedance,” U.S. patent application Ser. No. 10/364,434, entitled “Neural Stimulation System Providing Auto Adjustment of Stimulus Output as a Function of Sensed Pressure Changes,” U.S. Pat. No. 6,993,384, entitled “Apparatus and Method for Determining the Relative Position and Orientation of Neurostimulation Leads,” and U.S. patent application Ser. No. 11/096,483, entitled “Apparatus and Methods for Detecting Migration of Neurostimulation Leads,” which are expressly incorporated herein by reference.

It should be noted that rather than an IPG, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

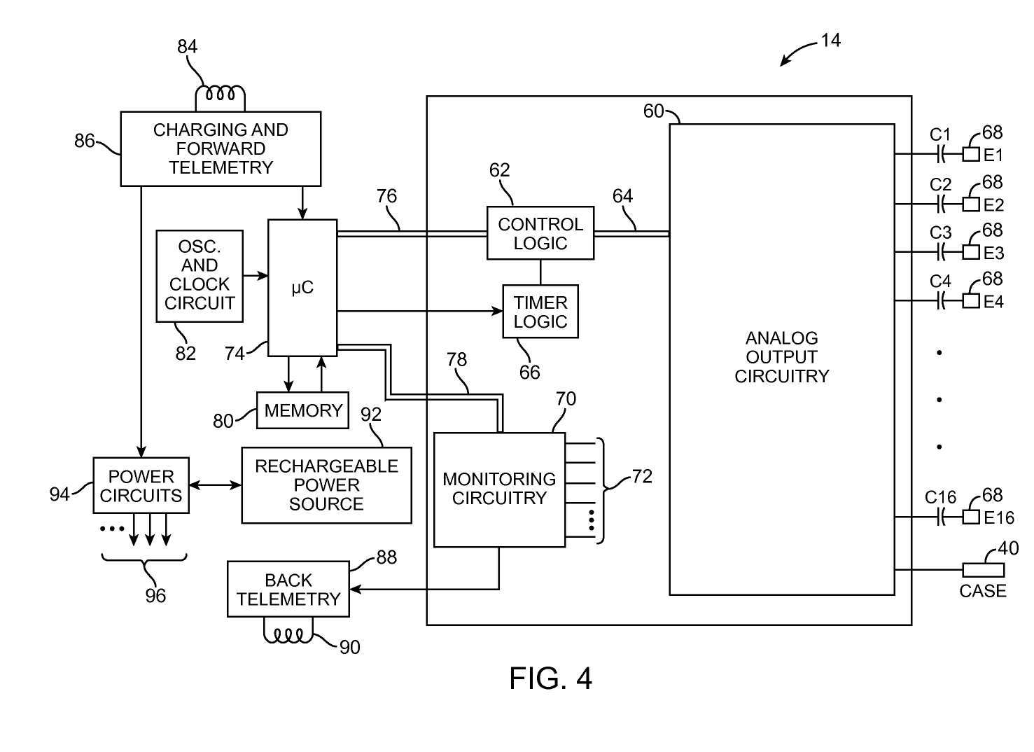

Turning next to FIG. 4, the main internal components of the IPG 14 will now be described. The IPG 14 includes stimulation output circuitry 60 configured for generating electrical stimulation energy in accordance with a defined pulsed waveform having a specified pulse amplitude, pulse rate, pulse width, pulse shape, and burst rate under control of control logic 62 over data bus 64. Control of the pulse rate and pulse width of the electrical waveform is facilitated by timer logic circuitry 66, which may have a suitable resolution, e.g., 10 μs. The stimulation energy generated by the stimulation output circuitry 60 is output via capacitors C1-C16 to electrical terminals 68 corresponding to the electrodes 26.

The stimulation output circuitry 60 may either comprise independently controlled current sources for providing stimulation pulses of a specified and known amperage to or from the electrical terminals 68, or independently controlled voltage sources for providing stimulation pulses of a specified and known voltage at the electrical terminals 68 or to multiplexed current or voltage sources that are then connected to the electrical terminals 68. The operation of this stimulation output circuitry, including alternative embodiments of suitable output circuitry for performing the same function of generating stimulation pulses of a prescribed amplitude and width, is described more fully in U.S. Pat. Nos. 6,516,227 and 6,993,384, which are expressly incorporated herein by reference.

The IPG 14 further comprises monitoring circuitry 70 for monitoring the status of various nodes or other points 72 throughout the IPG 14, e.g., power supply voltages, temperature, battery voltage, and the like. Notably, the electrodes 26 fit snugly within the epidural space of the spinal column, and because the tissue is conductive, electrical measurements can be taken from the electrodes 26 in order to determine the coupling efficiency between the respective electrode 26 and the tissue and/or to facilitate fault detection with respect to the connection between the electrodes 26 and the stimulation output circuitry 60 of the IPG 14. In the illustrated embodiment, the electrical measurements taken by the monitoring circuitry 70 may be any suitable measurement, e.g., an electrical impedance, an electrical field potential, or an evoked potential measurement. In alternative embodiments, the measurement may be non-electrical in nature, e.g., pressure, translucence, reflectance, or pH.

Further details discussing the measurement of electrical parameter data, such as electrode impedance, field potential, and evoked action potentials, as well as other parameter data, such as pressure, translucence, reflectance and pH (which can alternatively be used), to determine the coupling efficiency between an electrode and tissue are set forth in U.S. patent application Ser. No. 10/364,436, entitled “Neural Stimulation System Providing Auto Adjustment of Stimulus Output as a Function of Sensed Impedance,” U.S. patent application Ser. No. 10/364,434, entitled “Neural Stimulation System Providing Auto Adjustment of Stimulus Output as a Function of Sensed Pressure Changes,” U.S. Pat. No. 6,993,384, entitled “Apparatus and Method for Determining the Relative Position and Orientation of Neurostimulation Leads,” and U.S. patent application Ser. No. 11/096,483, entitled “Apparatus and Methods for Detecting Migration of Neurostimulation Leads,” which are expressly incorporated herein by reference.

The IPG 14 further comprises processing circuitry in the form of a microcontroller 74 that controls the control logic 62 over data bus 76, and obtains status data from the monitoring circuitry 70 via data bus 78. The microcontroller 74 additionally controls the timer logic 66. The IPG 14 further comprises memory 80 and an oscillator and clock circuit 82 coupled to the microcontroller 74. The microcontroller 74, in combination with the memory 80 and oscillator and clock circuit 82, thus comprise a microprocessor system that carries out a program function in accordance with a suitable program stored in the memory 80. Alternatively, for some applications, the function provided by the microprocessor system may be carried out by a suitable state machine.

Thus, the microcontroller 74 generates the necessary control and status signals, which allow the microcontroller 74 to control the operation of the IPG 14 in accordance with a selected operating program and parameters. In controlling the operation of the IPG 14, the microcontroller 74 is able to individually generate electrical pulses at the electrodes 26 using the stimulation output circuitry 60, in combination with the control logic 62 and timer logic 66, thereby allowing each electrode 26 to be paired or grouped with other electrodes 26, including the monopolar case electrode, and to control the polarity, amplitude, rate, and pulse width through which the current stimulus pulses are provided. The microcontroller 74 also controls information that is transmitted from and received by the IPG 14 via telemetry circuitry (described below).

As will be discussed in further detail below, the microcontroller 74 automatically adjusts stimulation energy conveyed from the stimulation output circuitry 60 in response to varying therapeutic environments and corresponding manual adjustments to the stimulation energy via the RC 16. The microcontroller 74 accomplishes this by associating user-defined stimulation parameter sets with measured parameters (discussed above with respect to the monitoring circuitry 70) indicative of the therapeutic environments in response to which the stimulation parameter sets were respectively defined to form reference templates, which can be in the memory 80 for subsequent usage in automatically adjusting the stimulation energy.

The IPG 14 further comprises an alternating current (AC) receiving coil 84 for receiving programming data (e.g., the operating program and/or stimulation parameters) from the RC 16 and/or CP 18 in an appropriate modulated carrier signal, and charging and forward telemetry circuitry 86 for demodulating the carrier signal it receives through the AC receiving coil 84 to recover the programming data, which programming data is then stored within the memory 80, or within other memory elements (not shown) distributed throughout the IPG 14. In addition to programming data, lead configuration information (presumably transmitted from an external control device separate from the RC 16 and/or CP 18) received via the AC receiving coil 84 and forward telemetry circuitry 86 can be stored in the memory 80.

The IPG 14 further comprises back telemetry circuitry 88 and an alternating current (AC) transmission coil 90 for sending informational data sensed through the monitoring circuitry 70 (including the measured data that can be used to generate the lead configuration information) to the RC 16 and/or CP 18. The back telemetry features of the IPG 14 also allow its status to be checked. For example, any changes made to the stimulation parameters are confirmed through back telemetry, thereby assuring that such changes have been correctly received and implemented within the IPG 14. Moreover, upon interrogation by the RC 16 and/or CP 18, all programmable settings, including lead configuration information, stored within the IPG 14 may be uploaded to the RC 16 and/or CP 18 via the telemetry circuitry 88 and AC transmission coil 90.

The IPG 14 further comprises a rechargeable power source 92 and power circuits 94 for providing the operating power to the IPG 14. The rechargeable power source 92 may, e.g., comprise a lithium-ion or lithium-ion polymer battery. The rechargeable battery 92 provides an unregulated voltage to the power circuits 94. The power circuits 94, in turn, generate the various voltages 96, some of which are regulated and some of which are not, as needed by the various circuits located within the IPG 14. The rechargeable power source 92 is recharged using rectified AC power (or DC power converted from AC power through other means, e.g., efficient AC-to-DC converter circuits) received by the AC receiving coil 84. To recharge the power source 92, an external charger (not shown), which generates the AC magnetic field, is placed against, or otherwise adjacent, to the patient's skin over the implanted IPG 14. The AC magnetic field emitted by the external charger induces AC currents in the AC receiving coil 84. The charging and forward telemetry circuitry 86 rectifies the AC current to produce DC current, which is used to charge the power source 92. While the AC receiving coil 84 is described as being used for both wirelessly receiving communications (e.g., programming and control data) and charging energy from the external device, it should be appreciated that the AC receiving coil 84 can be arranged as a dedicated charging coil, while another coil, such as coil 90, can be used for bi-directional telemetry.

It should be noted that the diagram of FIG. 4 is functional only, and is not intended to be limiting. Those of skill in the art, given the descriptions presented herein, should be able to readily fashion numerous types of IPG circuits, or equivalent circuits, that carry out the functions indicated and described. It should be noted that rather than an IPG for the neurostimulator, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

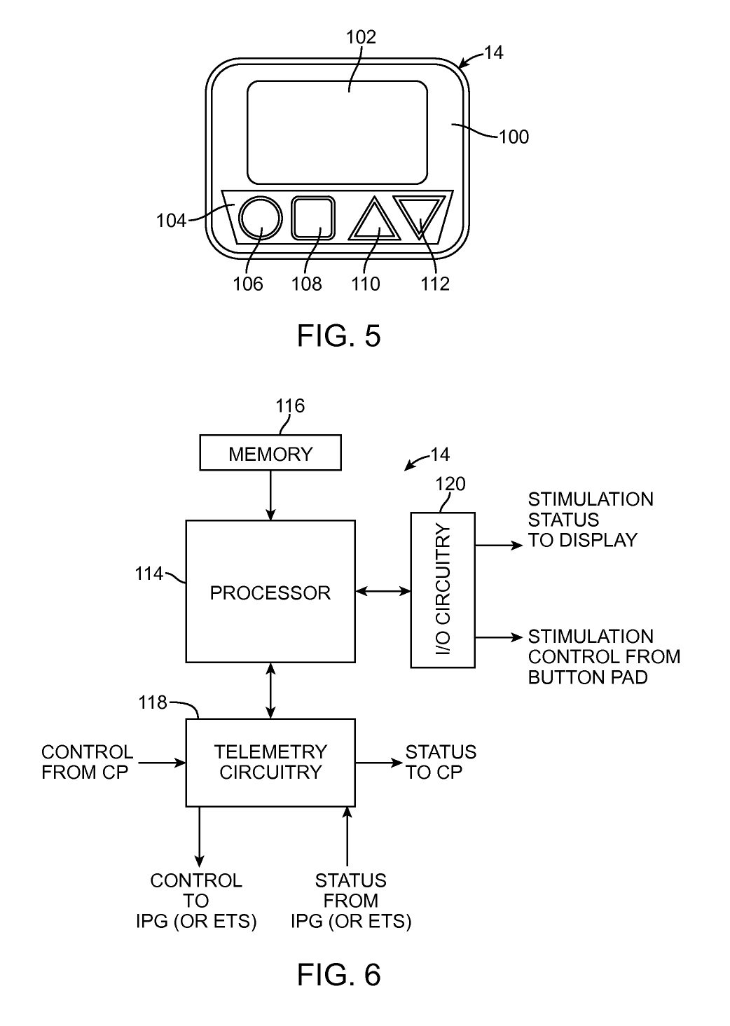

Referring now to FIG. 5, one exemplary embodiment of an RC 16 will now be described. As previously discussed, the RC 16 is capable of communicating with the IPG 14 or CP 18. The RC 16 comprises a casing 100, which houses internal componentry (including a printed circuit board (PCB)), and a lighted display screen 102 and button pad 104 carried by the exterior of the casing 100. In the illustrated embodiment, the display screen 102 is a lighted flat panel display screen, and the button pad 104 comprises a membrane switch with metal domes positioned over a flex circuit, and a keypad connector connected directly to a PCB. In an optional embodiment, the display screen 102 has touchscreen capabilities. The button pad 104 includes a multitude of buttons 106, 108, 110, and 112, which allow the IPG 14 to be turned ON and OFF, provide for the adjustment or setting of stimulation parameters within the IPG 14, and provide for selection between screens.

In the illustrated embodiment, the button 106 serves as an ON/OFF button that can be actuated to turn the IPG 14 ON and OFF. The button 108 serves as a select button that allows the RC 106 to switch between screen displays and/or parameters. The buttons 110 and 112 serve as up/down buttons that can be actuated to increase or decrease any of stimulation parameters of the pulse generated by the IPG 14, including pulse amplitude, pulse width, and pulse rate. For example, the selection button 108 can be actuated to place the RC 16 in a “Pulse Amplitude Adjustment Mode,” during which the pulse amplitude can be adjusted via the up/down buttons 110, 112, a “Pulse Width Adjustment Mode,” during which the pulse width can be adjusted via the up/down buttons 110, 112, and a “Pulse Rate Adjustment Mode,” during which the pulse rate can be adjusted via the up/down buttons 110, 112. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 6, the internal components of an exemplary RC 16 will now be described. The RC 16 generally includes a processor 114 (e.g., a microcontroller), memory 116 that stores an operating program for execution by the processor 114, and telemetry circuitry 118 for transmitting control data (including stimulation parameters and requests to provide status information) to the IPG 14 and receiving status information (including the measured electrical data) from the IPG 14 via link 34 (or link 32) (shown in FIG. 1), as well as receiving the control data from the CP 18 and transmitting the status data to the CP 18 via link 36 (shown in FIG. 1). The RC 16 further includes input/output circuitry 120 for receiving stimulation control signals from the button pad 104 and transmitting status information to the display screen 102 (shown in FIG. 5). As well as controlling other functions of the RC 16, which will not be described herein for purposes of brevity, the processor 114 generates new stimulation parameter sets in response to the user operation of the button pad 104. As will be described in further detail, these new stimulation parameter sets would then be transmitted to the IPG 14 via the telemetry circuitry 118 in order to train the automatic stimulation adjustment algorithm. Further details of the functionality and internal componentry of the RC 16 are disclosed in U.S. Pat. No. 6,895,280, which has previously been incorporated herein by reference.

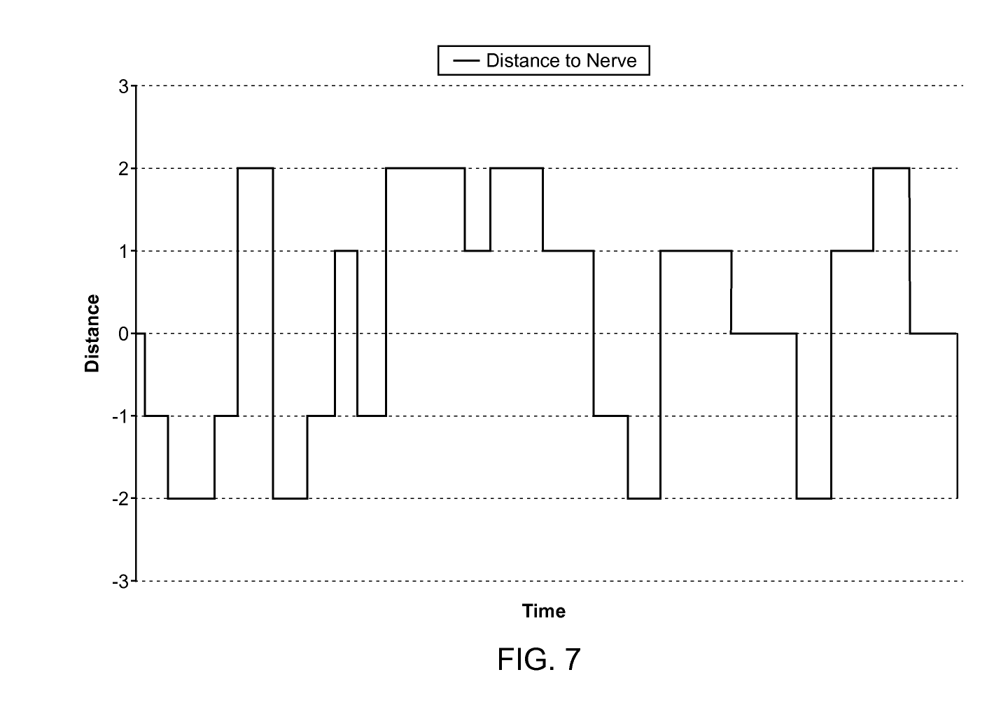

More significant to the present inventions, the IPG 14, in response to a change in the therapeutic environment, is capable automatically adjusting the stimulation energy in order to maintain efficacious therapy for the patient. As one example, the therapeutic environment may comprise the coupling efficiency between the stimulating electrodes 26 and the surrounding tissue, which may change as a result of postural changes, lead movement (acute and/or chronic), and scar tissue maturation. For example, with reference to FIG. 7, an exemplary plot of a coupling efficiency in the form of a distance between the activated electrodes and the target tissue to be stimulated will now be described. In this case, the distance is normalized, with the distance of 0 representing the normalized distance between the activated electrodes and the target tissue to which the stimulation energy was initially optimized. As shown, the normalized distance varies from 2 to −2 over time.

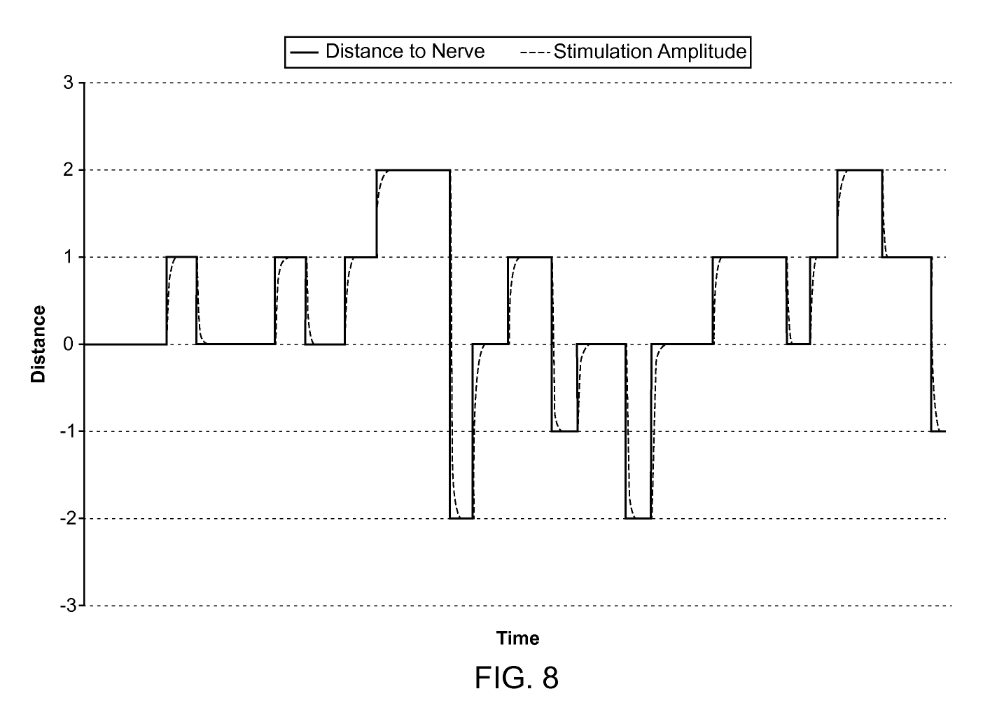

In the case where the coupling efficiency between the activated electrodes and the target tissue significantly changes, adjustments in the stimulation energy may need to be made in order to maintain an efficacious and comfortable therapy. This is typically accomplished by manually adjusting the stimulation energy in response to the changes in the coupling efficiency. For example, with reference to FIG. 8, the patient may manually adjust the stimulation energy via the control buttons 110, 112 (shown in FIG. 5) in response to changes in the normalized distance between the activated electrodes and the target tissue. In particular, as the normalized distance between the activated electrodes and the target tissue increases, the intensity of the stimulation energy may be increased by the patient to maintain efficacious therapy, and if the normalized distance between the activated electrodes and the target tissue decreases, the intensity of the stimulation energy may be decreased by the patient to maintain comfortable therapy. For purposes of visualizing the manual tracking between coupling efficiency and the user adjustments in the stimulation energy, the intensity of the user adjusted stimulation energy is shown to match the distance between the stimulating electrodes and the target tissue.

While the change in the therapeutic environment has been expressed in terms of coupling efficiency, and the user adjustments in the stimulation energy have been described as an adjustment in the intensity, other types of therapeutic environments and adjustments in stimulation energy are possible. For example, the therapeutic environment may be the status of the dysfunction being treated (e.g., the dysfunction suffered by the patient may be more symptomatic during one time of the day than another time of the day, or medication taken by the patient may either beneficially or adversely affect the status of the dysfunction), and the user adjustments in the stimulation energy may be adjustments in the pulse rate, electrode combinations, etc. It should also be appreciated that the intensity of the stimulation energy may be adjusted by modifying the pulse amplitude and/or the pulse width.

Advantageously, the RC 16 is capable of continuously training this automated stimulation adjustment algorithm in the IPG 14 in response to the manual adjustment of the stimulation energy by the patient (or otherwise the user) as the therapeutic environment changes. In this manner, the user need not have to manually adjust the stimulation energy in response a change in a therapeutic environment on which the automated stimulation adjustment algorithm has been trained. At any time, the user may manually make the stimulation energy adjustments (e.g., by generating user-defined stimulation parameter sets via the control buttons 56-62) in response to changes in the therapeutic environment, but over time, the IPG 14 will associate the manual stimulation energy adjustments from the RC 16 with the different therapeutic environments, and based on these associations, the IPG 14 may recognize the changes in the therapeutic environment (e.g., by acquiring measured parameters indicative of the therapeutic environment changes) and automatically adjust the stimulation energy to restore an efficacious and comfortable stimulation therapy for the patient.

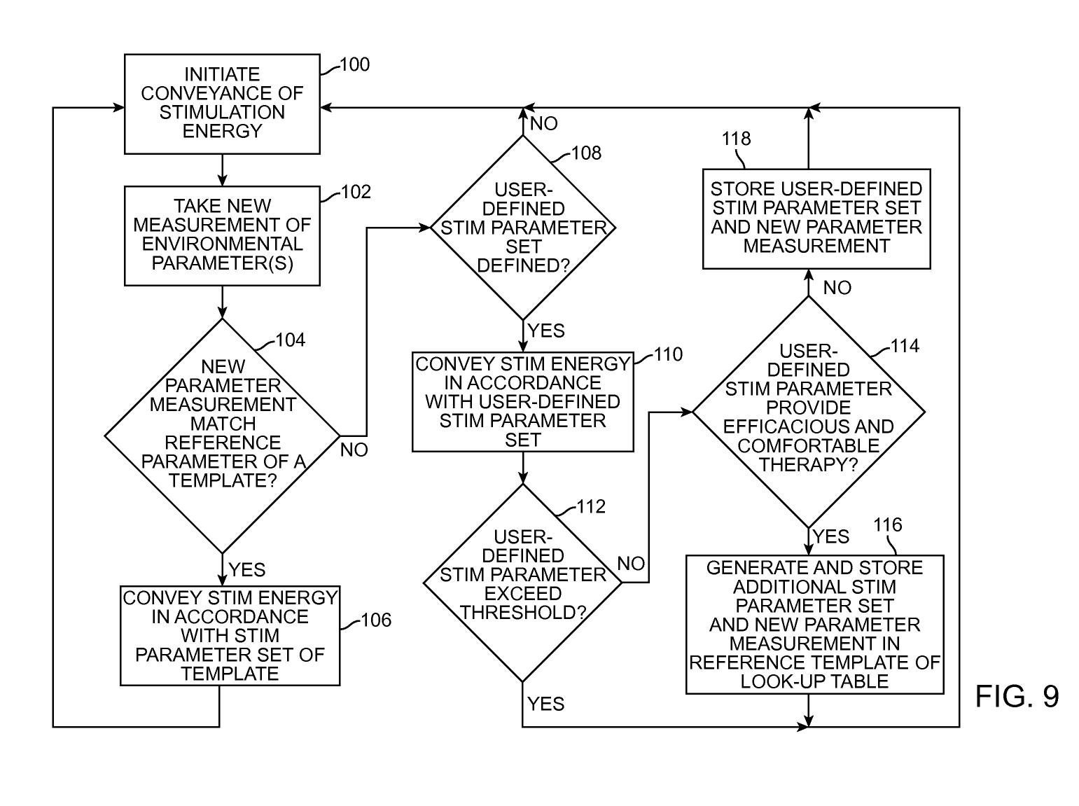

Referring to FIG. 9, one exemplary method of training the IPG 14 to automatically adjust the stimulation energy conveyed in response to various therapeutic environments will now be described. Initially, there may be no reference templates (i.e., corresponding reference stimulation parameter sets and reference parameters indicative of the therapeutic environment) stored in the IPG 14. For example, the IPG 14 may be provided to the patient without reference templates. Alternatively, the IPG 14 may be provided to the patient with a limited number of reference templates that are stored in a look-up table. These reference templates could even be provided in the RC 16 or CP 18 and downloaded into the IPG 14 for use. In either event, the number of reference templates generated in the clinician's office will not take into account all possible therapeutic environments, especially those that would evolve over a period of time.

First, the RC 16 is operated to initiate the conveyance of stimulation energy from the IPG 14 by, e.g., actuating the button 106 (step 200). Next, a new measurement of one or more environmental parameters indicative of a change in a therapeutic environment is taken by the IPG 14 (step 202). The environmental parameter that is measured will depend on the relevant therapeutic environment. For example, if the relevant therapeutic environment is coupling efficiency between the stimulation electrodes and the surrounding tissue, an impedance, field potential, evoked potential, pressure, translucence, reflectance, or pH may be measured. If the relevant therapeutic environment is a time of day, the IPG 14 may measure the time via an internal clock. If the relevant therapeutic environment is a status of the dysfunction that is treated, a biological signal or medication schedule input to the IPG 14 by the patient can be measured.

Next, the IPG 14 determines whether the new parameter measurement matches one of the reference parameters of the templates previously stored within the look-up table (to the extent that a previous template has been stored) (step 204). In the illustrated embodiment, the IPG 14 may determine whether there is an identical match between the new parameter measurement and one of the reference parameters. In an alternative embodiment, the IPG 14 may determine whether there is an approximate match between the new parameter measurement and one of the reference parameters. For example, the IPG 14 may compare data points of the measured parameter with data points of the reference parameter using a comparison function (e.g., a correlation coefficient function, such as a Pearson Correlation Coefficient function, sum of squared differences function, cross-correlation functions, wavelet functions, associated matching measures, etc.) and determine a match based on this comparison function (e.g., by comparing a resultant value of the comparison function to a threshold value). Examples of comparison functions are described in U.S. patent application Ser. No. 12/941,657, entitled “Automatic Lead Identification Using Electrical Field Fingerprinting,” which is expressly incorporated herein by reference.

If a match is determined at step 204, the IPG 14 conveys electrical stimulation energy to the patient in accordance with the stimulation parameter set obtained from the look-up table (i.e., the stimulation parameter set corresponding to the matching reference parameter) (step 206), and then returns to step 202 to take another new measurement of the environmental parameter(s). In an optional embodiment, the IPG 14 will only make downward adjustments in stimulation energy intensity, and would require the user to make any upward adjustment in the stimulation energy intensity manually. If a match is not determined at step 204, the IPG 14 determines whether a user-defined stimulation parameter set has been generated (in effect, by the user manipulating controls 106-122 on the RC 16) (step 208). If a user-defined stimulation parameter set has not been generated at step 208, the IPG 14 returns to step 202 to take another new measurement of the environmental parameter(s). If a user-defined stimulation parameter set has been generated at step 208, the IPG 14 conveys electrical stimulation energy to the patient in accordance with the user-defined stimulation parameter set (step 210).

The IPG 14 then generates and stores an additional reference template in the look-up table based on the user-defined stimulation parameter set, which in the illustrated embodiment, is only performed under certain conditions. In particular, the IPG 14 first compares the user-defined stimulation parameter set to a safety threshold (step 212), and if the threshold is exceeded, the IPG 14 does not generate and store an additional reference template within the look-up table, but instead, returns to step 202 to take another new measurement of the environmental parameter(s). For example, it may be deemed that a stimulation intensity greater than a certain amount could cause tissue damage or pain to the patient. In this case, if the user adjusts the stimulation energy to an intensity that exceeds the threshold intensity, the IPG 14 will not generate a reference template based on the user-defined stimulation parameter set.

If, at step 212, the threshold is not exceeded, the IPG 14 confirms whether the user-defined stimulation parameter set, in fact, provides efficacious and comfortable therapy for the patient (step 214). In particular, even though the patient, in response to a changing therapeutic environment, may adjust the stimulation energy for the purpose of maintaining efficacious and comfortable therapy, it may not initially be known whether the adjusted stimulation energy will, in fact, provide that efficacious and comfortable therapy. For example, the patient may need to manually adjust the stimulation energy several times in order to find an efficacious or comfortable stimulation parameter set, or the therapeutic environment may change so quickly that it cannot be easily tracked by the user-defined stimulation energy.

In the illustrated embodiment, the IPG 14 makes this determination by determining whether the measurement of the environmental parameter(s) and the user-defined stimulation parameter set are stable over time (i.e., if they both remain the same over a significant period of time, it can be assumed that efficacious and comfortable therapy has been achieved; otherwise, the user would modify the stimulation energy). The time period over which the measurement of the environmental parameters(s) and the user-defined stimulation parameter set are determined to be stable may be defined by a fixed time or a predetermined number of measurements.

The IPG 14 may determine stability of the stimulation by, e.g., comparing the new environmental parameter measurement with parameters that were previously measured during stimulation performed in accordance with the same user-defined stimulation parameter, and if the new environmental parameter measurement matches a predetermined number of the same environmental parameter measurements, determining that stability in stimulation, and thus efficacious and comfortable therapy, has been achieved.

If the IPG 14 confirms, at step 214, that the user-defined stimulation parameter set does, in fact, provides efficacious and comfortable therapy for the patient, the RC 16 generates an additional reference stimulation parameter set from the user-defined stimulation parameter set, and stores the new environmental parameter measurement in association with the additional reference stimulation parameter set within the look-up table (step 216). In the illustrated embodiment, the additional reference stimulation parameter set is identical to the user-defined stimulation parameter set. In alternative embodiments, the additional reference stimulation parameter set may be a modification of the user-defined stimulation parameter set.

If the IPG 14 cannot confirm, at step 214, that the user-defined stimulation parameter set does, in fact, provides efficacious and comfortable therapy for the patient, the IPG 14 stores the user-defined stimulation parameter set and the measured environmental parameter(s) (step 218), but not in the look-up table. Rather, the stored user-defined stimulation parameter set and measured environmental parameter(s) will be stored for use in determining whether subsequent user-defined stimulation parameter sets provide efficacious and comfortable therapy for the patient. The IPG 14 then returns to step 202 to take another new measurement of the environmental parameter(s).

As this process continues, the automated stimulation adjustment algorithm is trained for each therapeutic environment. Once training is complete for all therapeutic environments, the automated stimulation adjustment algorithm can take over and no user adjustment of the stimulation is necessary as long as the relationship between the therapeutic environments and the effective and comfortable stimulation remains the same. If, however, the user were to make adjustments in the stimulation for a particular therapeutic environment, the training of the automated stimulation adjustment algorithm for this therapeutic environment would be reset (e.g., by deleting the reference template associated with the therapeutic environment) and retraining the automated stimulation adjustment algorithm for the therapeutic environment would occur. Alternatively, rather than resetting the training of the automated stimulation adjustment algorithm for that therapeutic environment (i.e., training of the algorithm is performed anew without any use of previous data), the existing user-defined stimulation parameter set is simply taken into account along with the last number of user-defined stimulation parameter sets in a continuous training algorithm. More alternatively, the existing user-defined stimulation parameter set can be compared to the user-defined stimulation parameter set in the reference template, and if the difference is greater than a threshold value, the training of the automated stimulation adjustment algorithm for the therapeutic environment can be performed anew without the use of any previous data, and if the difference is less than the threshold value, the existing user-defined stimulation parameter set can be taken into account with the last number of user-defined stimulation parameter sets in a continuous training algorithm.

It should be appreciated that if the stimulation parameter sets are intensity-based, they may include both pulse amplitude and pulse width. For example, for each pulse width at which the automated stimulation adjustment algorithm is trained, a plurality of stimulation parameter sets, each including different pulse amplitudes, may need to be generated. In this manner, a two-dimensional array of stimulation parameter sets would be required to train the automated stimulation adjustment algorithm, with one dimension being the pulse width, and the other dimension being the pulse amplitude.

Alternatively, rather than using a two-dimensional array of stimulation parameter sets, a strength-duration curve, which defines the intensity of the stimulation as a function of pulse amplitude and pulse width, as described in U.S. patent application Ser. No. 11/553,447, entitled “Method of Maintaining Intensity Output While Adjusting Pulse Width or Amplitude,” which is expressly incorporated herein by reference, can be utilized in a manner that would only require the automated stimulation adjustment algorithm to be trained over a single pulse width. For example, if the automated stimulation energy algorithm is initially trained over a first pulse width, and then the user pulse width is subsequently modified by the user, the reference stimulation parameters that were generated with the first pulse width can still be used to automatically adjust the stimulation energy at the new pulse width. For example, for each reference stimulation parameter set initially generated at the first pulse width, the intensity of the stimulation can be assumed from the pulse amplitude and pulse width, and then, at the new pulse width, the pulse amplitude required to maintain that same stimulation intensity in that reference stimulation parameter set can be inferred in order to adjust the stimulation energy to the proper amplitude. Other stimulation parameters that could be included in the stimulation parameter set are rate, electrode configuration and electrode polarity.

While the illustrated embodiment has been described as automatically switching the IPG 14 between an automated stimulation adjustment mode and a training mode, it should be appreciated that one or more control buttons (not shown) can be provided on the RC 16 for manually switching the IPG 14 between these two modes. Furthermore, additional control buttons 16 can be provided on the RC 16 for adjusting the aggressiveness of the training mode (e.g., adjusting the time needed to confirm that a particular user-defined stimulation parameter set provides efficacious and comfortable therapy for a given therapeutic environment). Additional control buttons 16 can also be provided on the RC 16 for adjusting the absolute or relative amplitude that the automated stimulation adjustment algorithm can increase the amplitude of the stimulation energy or move the stimulation field.

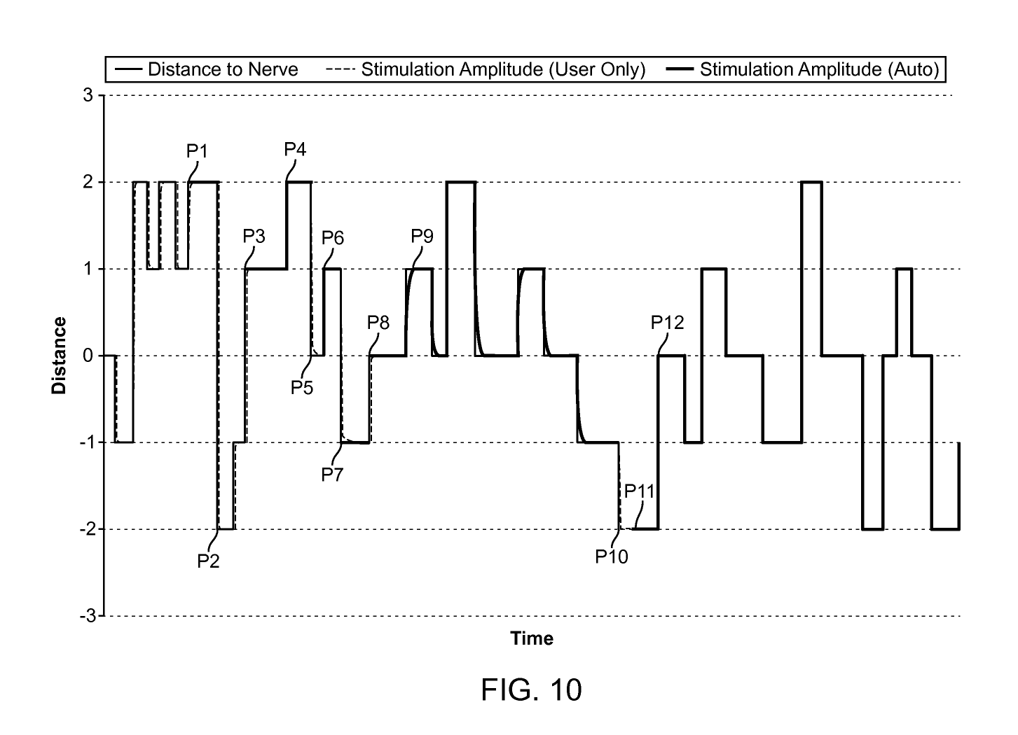

Referring now to FIG. 10, an exemplary training session for an automated stimulation adjustment algorithm will be described. In this case, the manual adjustments to the intensity of the stimulation energy, to the extent that they are made, and the changes in the coupling efficiency in the form of the normalized distance between the activated electrodes and the target tissue, are shown to be the same as that shown in FIG. 8. Initially, the normalized distance is zero (the distance at which the stimulation energy was initially optimized). As the normalized distance transitions from −1 to +2, the user adjusts the intensity of the stimulation energy to maintain efficacious and comfortable therapy during a training mode. Once the automated stimulation adjustment algorithm is trained for a particular normalized distance, the IPG 14 may be operated in an automated stimulation adjustment mode. During any training mode, the automated stimulation adjustment mode is preferably disabled.

In the exemplary case illustrated in FIG. 10, the IPG 14 remains in this training mode until the beginning of the third transition to the +2 normalized distance (point P1) where the RC 16 determines that the training on the +2 normalized distance has been completed. That is, the IPG 14 has accumulated enough data at the +2 normalized distance (obtained from the previous first and second transitions to the +2 normalized distance) to determine that the manually adjusted stimulation energy is, in fact, efficacious and comfortable at that distance. Thus, the IPG 14 enables the automated stimulation adjustment mode, which maintains the stimulation energy at the same level until the transition to the −2 normalized distance (point P2). At this point, the IPG 14 disables the automated stimulation adjustment mode, and the IPG 14 is placed into the training mode, with the patient manually adjusting the intensity of the stimulation energy to maintain efficacious and comfortable therapy. As the normalized distance transitions from −2 to −1, and then from −1 to +1, the IPG 14 remains in the training mode to gather additional data in response to the manual adjustments in the stimulation energy intensity.

At the beginning of the third transition to the +1 normalized distance (point P3), the IPG 14 has accumulated enough data at the +1 normalized distance (obtained from the previous first and second transitions to the +1 normalized distance) to determine that the manually adjusted stimulation energy is, in fact, efficacious and comfortable at that distance. Thus, the IPG 14 enables the automated stimulation adjustment mode, which maintains the stimulation energy at the same level until the normalized distance transitions to +2 (point P4). When the normalized distance transitions from +1 to +2, the IPG 14 automatically adjusts the stimulation energy to the intensity corresponding to the previously trained +2 normalized distance.

The IPG 14 maintains the stimulation energy at the same level until the transition to the 0 normalized distance (point P5). At this point, the IPG 14 disables the automated stimulation adjustment mode, and the IPG 14 is placed into the training mode, with the patient manually adjusting the intensity of the stimulation energy to maintain efficacious and comfortable therapy. When the normalized distance transitions from 0 to +1 (point P6), the IPG 14 enables the automated stimulation adjustment mode, which automatically adjusts the stimulation energy to the intensity corresponding to the previously trained +1 normalized distance.

At the beginning of the third transition to the −1 normalized distance (point P7), the IPG 14 is placed into the training mode, with the patient manually adjusting the intensity of the stimulation energy to maintain efficacious and comfortable therapy. At this point, the IPG 14 has accumulated enough data at the −1 normalized distance (obtained from the previous first and second transitions to the −1 normalized distance) to determine that the manually adjusted stimulation energy is, in fact, efficacious and comfortable at that distance. Thus, the IPG 14 enables the automated stimulation adjustment mode, which maintains the stimulation energy at the same level until the transition to the 0 normalized distance (point P8).

At the beginning of the third transition to the 0 normalized distance, the IPG 14 is placed into the training mode, with the patient manually adjusting the intensity of the stimulation energy to maintain efficacious and comfortable therapy. At this point, the RC 16 has accumulated enough data at the 0 normalized distance (obtained from the previous first and second transitions to the 0 normalized distance) to determine that the manually adjusted stimulation energy is, in fact, efficacious and comfortable at that distance. Thus, the IPG 14 enables the automated stimulation adjustment mode, which maintains the stimulation energy at the same level until the transition to the +1 normalized distance (point P9). When the normalized distance transitions from 0 to +1, the IPG 14 automatically adjusts the stimulation energy to the intensity corresponding to the previously trained +1 normalized distance.

The IPG 14 continues to automatically adjust the stimulation energy to the intensities corresponding to the previously trained normalized distances until the transition to the −2 normalized distance (point P10). At this point, the IPG 14 disables the automated stimulation adjustment mode, and the IPG 14 is placed into the training mode, with the patient manually adjusting the intensity of the stimulation energy to maintain efficacious and comfortable therapy.

At the beginning of the second transition to the −2 normalized distance, the IPG 14 is placed into the training mode, with the patient manually adjusting the intensity of the stimulation energy to maintain efficacious and comfortable therapy. At a certain point after the transition to the −2 normalized distance (point P11), the IPG 14 has accumulated enough data at the −2 normalized distance (obtained from the previous first transition and part of the second transition to the −2 normalized distance) to determine that the manually adjusted stimulation energy is, in fact, efficacious and comfortable at that distance. Thus, the IPG 14 enables the automated stimulation adjustment mode, which maintains the stimulation energy at the same level until the transition to the 0 normalized distance (point P12). The IPG 14 continues to automatically adjust the stimulation energy to the intensities corresponding to the previously trained normalized distances until the transition to a normalized distance that has not been previously trained or needs to be retrained.

Although the foregoing techniques have been described as being implemented in the IPG 14, it should be noted that this technique may be alternatively or additionally implemented in the RC 16 or CP 18 with stimulation parameters initiated via telemetry to the IPG 14. Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A neurostimulator, comprising:
input/output circuitry configured for receiving a user-defined stimulation parameter set from an external control device;
stimulation output circuitry configured for conveying electrical stimulation energy;
monitoring circuitry configured for acquiring a new measurement of least one environmental parameter indicative of a change in a therapeutic environment;
memory configured for storing a plurality of reference measurements and a plurality of reference stimulation parameter sets respectively associated with the reference measurements; and
a controller configured for determining whether the new measurement matches one of the stored reference measurements, if a match is determined, instructing the stimulation output circuitry to convey electrical stimulation energy in accordance with the stimulation parameter set corresponding to the matching reference measurement, and if a match is not determined, instructing the stimulation output circuitry to convey electrical stimulation energy in accordance with the user-defined stimulation parameter set, defining another reference stimulation parameter set based on the user-defined stimulation parameter set, and storing the new measurement as an additional reference measurement in the memory in association with the additional reference stimulation parameter set.

2. The neurostimulator of claim 1, wherein the controller is configured for defining the user-defined stimulation parameter set as the additional reference stimulation parameter set.

3. The neurostimulator of claim 2, wherein the controller is further configured for comparing the user-defined stimulation parameter set to a threshold, and defining the user-defined stimulation parameter as the additional reference stimulation parameter set only if the user-defined stimulation parameter set does not exceed the threshold.

4. The neurostimulator of claim 1, wherein the monitoring circuitry is further configured for acquiring a plurality of new measurements, each of least one environmental parameter indicative of a change in a therapeutic environment, wherein the controller is configured for storing the additional reference stimulation parameter set in the memory only if the plurality of new measurements are substantially the same during the conveyance of the electrical stimulation in accordance with the user-defined stimulation parameter set.

5. The neurostimulator of claim 4, wherein the plurality of new measurements is a predetermined number.

6. The neurostimulator of claim 1, wherein the at least one environmental parameter comprises at least one of an impedance, field potential, evoked potential, pressure, translucence, reflectance, pH, acceleration, chemical, neural recordings, and time of day.

7. The neurostimulator of claim 1, wherein each of the reference stimulation parameter sets comprises at least one of a stimulation energy intensity and an electrode combination.

8. The neurostimulator of claim 1, further comprising a housing containing the input/output circuitry, stimulation output circuitry, memory, and controller.

9. A neurostimulation system, comprising:
an external control device configured for allowing a user to define a stimulation parameter set; and
a neurostimulator configured for acquiring a new measurement of least one environmental parameter indicative of a change in a therapeutic environment, storing a plurality of reference measurements and a plurality of reference stimulation parameter sets respectively associated with the reference measurements, and determining whether the new measurement matches one of the stored reference measurements, if a match is determined, conveying electrical stimulation energy in accordance with the stimulation parameter set corresponding to the matching reference measurement, and if a match is not determined, conveying electrical stimulation energy in accordance with the user-defined stimulation parameter set, defining another reference stimulation parameter set based on the user-defined stimulation parameter set, and storing the new measurement as an additional reference measurement in the memory in association with the additional reference stimulation parameter set.

10. The neurostimulation system of claim 9, wherein the neurostimulator is configured for defining the user-defined stimulation parameter set as the additional reference stimulation parameter set.

11. The neurostimulation system of claim 10, wherein the neurostimulator is further configured for comparing the user-defined stimulation parameter set to a threshold, and defining the user-defined stimulation parameter as the additional reference stimulation parameter set only if the user-defined stimulation parameter set does not exceed the threshold.

12. The neurostimulation system of claim 9, wherein the neurostimulator is further configured for receiving a plurality of new measurements, each of least one environmental parameter indicative of a change in a therapeutic environment, and storing the additional reference stimulation parameter set only if the plurality of new measurements are substantially the same during the conveyance of the electrical stimulation in accordance with the user-defined stimulation parameter set.

13. The neurostimulation system of claim 12, wherein the plurality of new measurements is a predetermined number.

14. The neurostimulation system of claim 9, wherein the at least one environmental parameter comprises at least one of an impedance, field potential, evoked potential, pressure, translucence, reflectance, pH, acceleration, chemical, neural recordings, and time of day.

15. The neurostimulation system of claim 9, wherein each of the reference stimulation parameter sets comprises at least one of a stimulation energy intensity and an electrode combination.

16. A method of operating a neurostimulation system to provide therapy to a patient via electrical stimulation energy, the neurostimulation system configured for storing a plurality of reference measurements and a plurality of reference stimulation parameter sets respectively associated with the reference measurements, the method comprising:
taking a new measurement of least one environmental parameter indicative of a change in a therapeutic environment;
determining whether the new measurement matches one of the stored reference measurements;
if a match is determined, conveying electrical stimulation energy from the neurostimulation system to the patient in accordance with the stimulation parameter set corresponding to the matching reference measurement; and
if a match is not determined, conveying electrical stimulation energy from the neurostimulation system to the patient in accordance with a user-defined stimulation parameter set, defining another reference stimulation parameter set based on the user-defined stimulation parameter set, and storing the new measurement as an additional reference measurement in association with the additional reference stimulation parameter set.

17. The method of claim 16, wherein the user-defined stimulation parameter set is defined as the additional reference stimulation parameter set.

18. The method of claim 17, further comprising comparing the user-defined stimulation parameter set to a threshold, wherein the user-defined stimulation parameter is only defined as the additional reference stimulation parameter set if the user-defined stimulation parameter set does not exceed the threshold.

19. The method of claim 16, further comprising taking a plurality of new measurements, each of least one environmental parameter indicative of a change in a therapeutic environment, wherein the additional reference stimulation parameter set is only stored if the plurality of new measurements are substantially the same during the conveyance of the electrical stimulation in accordance with the user-defined stimulation parameter set.

20. The method of claim 19, wherein the plurality of new measurements is a predetermined number.

21. The method of claim 16, wherein the at least one environmental parameter comprises at least one of an impedance, field potential, evoked potential, pressure, translucence, reflectance, pH, acceleration, chemical, neural recordings, and time of day.

22. The method of claim 16, wherein each of the reference stimulation parameter sets comprises at least one of a stimulation energy intensity and an electrode combination.

23. The method of claim 16, wherein the therapy is the treatment of chronic pain.
